(19) United States
(12) Patent Application Publication
(10) Pub. No.: US 2022/0331353 A1
(43) Pub. Date: Oct. 20, 2022

(54) SYNERGISTIC COMPOSITIONS AND METHODS TO INCREASE VASCULAR NITRIC OXIDE TO TREAT ENDOTHELIAL DYSFUNCTION AND RELATED CONDITIONS

(71) Applicant: Calroy Health Sciences, LLC, Scottsdale, AZ (US)

(72) Inventors: Chen Chen, Scottsdale, AZ (US); Edward Hoyt, Scottsdale, AZ (US); Kevin Chen, Scottsdale, AZ (US); Derek Chen, Scottsdale, AZ (US)

(21) Appl. No.: 17/735,106

(22) Filed: May 2, 2022

Related U.S. Application Data
(63) Continuation-in-part of application No. 17/680,927, filed on Feb. 25, 2022.
(60) Provisional application No. 63/153,867, filed on Feb. 25, 2021.

(51) Int. Cl. A61K 33/00; A61K 47/22; A61K 47/02; A61P 9/12

(52) U.S. Cl. CPC A61K 33/00; A61K 47/22; A61K 47/02; A61P 9/12

(57) ABSTRACT

A method of treating endothelial dysfunction in a subject can include: identifying damage in an endothelial glycocalyx (EGX) of the subject, and administering to the subject a combination of a nitric oxide (NO) precursor and a hydrogen sulfide (H₂S) precursor in an amount and at a frequency sufficient to stabilize and reverse damage in the EGX.

A therapeutic composition for treating endothelial dysfunction can include a combination of a nitric oxide (NO) precursor and a hydrogen sulfide (H₂S) precursor in an amount sufficient to sustain an increase of bioavailable NO for the treatment of endothelial dysfunction, and a pharmaceutically acceptable carrier.

### RELATED APPLICATION

This application is a continuation-in-part of U.S. application Ser. No. 17/680,927, filed Feb. 25, 2022, which claims priority to U.S. Provisional Patent Application No. 63/153,867, filed Feb. 25, 2021 which are each incorporated herein by reference.

### BACKGROUND

Overproduction of reactive oxygen species (ROS) under pathophysiologic conditions may be involved in the development of cardiovascular diseases (CVD). These ROS can be released from nicotinamide adenine dinucleotide (phosphate) oxidase, xanthine oxidase, lipoxygenase, mitochondria, or the uncoupling of nitric oxide synthase in vascular cells. ROS may mediate various signaling pathways that underlie vascular inflammation in atherogenesis: from the initiation of fatty streak development through lesion progress to ultimate plaque rupture. Various animal models of oxidative stress support the notion that ROS is involved in atherosclerosis and other cardiovascular diseases, and some human investigations also support the oxidative stress hypothesis of atherosclerosis.

Endothelial dysfunction (ED) is at the center of CVD. Many risk factors of heart disease cause damage to the endothelium and lead to ED. The common mechanism behind the endothelial damage is oxidative stress and the resulting inflammation and immune response. When damaged, the endothelium loses its capacity to produce nitric oxide (NO). The reduction of vascular NO leads to a further increase of oxidative stress in the system. This vicious cycle and the resulting loss of homeostatic balance between NO and ROS are the fundamental cause of ED.

Many biological pathways and processes are involved in the control and metabolism of NO and ROS inside the body. Various products are available to target some of them separately.

### BRIEF DESCRIPTION OF THE DRAWINGS

Features and advantages of the invention will be apparent from the detailed description that follows, and which taken in conjunction with the accompanying figures together illustrate features of the invention. It is understood that the figures merely depict exemplary embodiments and are therefore, not to be considered limiting in scope.

FIG. 1 is a graph of saliva nitrite (NO₂) over time in accordance with Example 4.

FIG. 2 is a graph of blood pressure over time in accordance with Example 4.

FIG. 3 is a graph of diastolic blood pressure by subgroup over time in accordance with Example 4.

### SUMMARY

A method of treating endothelial dysfunction (ED) in a subject can optionally include identifying endothelial glycocalyx (EGX) and/or endothelial dysfunction of the subject. The method can further include administering to the subject a combination of a nitric oxide (NO) precursor and a hydrogen sulfide (H₂S) precursor in an amount and at a frequency sufficient to restore and sustain an optimal or improved NO level and endothelial function.

A therapeutic composition for treating endothelial dysfunction can include a combination of a nitric oxide (NO) precursor and a hydrogen sulfide (H₂S) precursor in an amount sufficient to treat endothelial dysfunction.

A therapeutic composition for treating endothelial dysfunction can further include one or more antioxidants to deactivate superoxide and its downstream ROS to increase NO availability.

A therapeutic composition for treating endothelial dysfunction can further include an endothelial glycocalyx regenerating compound (eGRC) to repair and restore EGX to support optimal or improved NO synthesis by endothelial nitric oxide synthase (eNOS) and antioxidant defense against endothelial damage.

A therapeutic composition for treating endothelial dysfunction can further include one or more cofactors to optimize the production, transportation, storage and release of nitric oxide in the body. The therapeutic composition can further include a pharmaceutically acceptable carrier.

An oral dosage form can include a combination of a nitric oxide (NO) precursor and a hydrogen sulfide (H₂S) precursor in an amount sufficient to sustain a long-lasting effect to treat endothelial dysfunction. The oral dosage form can further include a pharmaceutically acceptable carrier.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

### Definitions

In describing and claiming the present invention, the following terminology will be used.

The singular forms “a,” “an,” and “the” include plural referents unless the context clearly dictates otherwise. Thus, for example, reference to “a cofactor” includes reference to one or more of such materials and reference to “subjecting” refers to one or more such steps.

As used herein, the term “about” is used to provide flexibility and imprecision associated with a given term, metric or value. The degree of flexibility for a particular variable can be readily determined by one skilled in the art. However, unless otherwise enunciated, the term “about” generally connotes flexibility of less than 2%, and most often less than 1%, and in some cases less than 0.01%.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

The term “dosage unit” or “dose” are understood to mean an amount of an active agent that is suitable for administration to a subject in order achieve or otherwise contribute to a therapeutic effect. In some examples, a dosage unit can refer to a single dose that is capable of being administered to a subject or patient, and that may be readily handled and packed, remaining as a physically and chemically stable unit dose.

As used herein, a “dosing regimen” or “regimen” such as “treatment dosing regimen,” or a “prophylactic dosing regimen” refers to how, when, how much, and for how long a dose of an active agent or composition can or should be administered to a subject in order to achieve an intended treatment or effect.

As used herein, the terms “treat,” “treatment,” or “treating” refers to administration of a therapeutic agent to subjects who are either asymptomatic or symptomatic. In other words, “treat,” “treatment,” or “treating” can be to reduce, ameliorate or eliminate symptoms associated with a condition present in a subject, or can be prophylactic, (i.e. to prevent or reduce the occurrence of the symptoms in a subject). Such prophylactic treatment can also be referred to as prevention of the condition.

As used herein, the terms “therapeutic agent,” “active agent,” and the like can be used interchangeably and refer to an agent that can have a beneficial or positive effect on a subject when administered to the subject in an appropriate or effective amount.

The phrase “effective amount,” “therapeutically effective amount,” or “therapeutically effective rate(s)” of an active ingredient refers to a substantially non-toxic, but sufficient amount or delivery rates of the active ingredient, to achieve therapeutic results in treating a disease or condition for which the drug is being delivered. It is understood that various biological factors may affect the ability of a substance to perform its intended task. Therefore, an “effective amount,” “therapeutically effective amount,” or “therapeutically effective rate(s)” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a subjective decision. The determination of a therapeutically effective amount or delivery rate is well within the ordinary skill in the art of pharmaceutical sciences and medicine.

As used herein, “formulation” and “composition” can be used interchangeably and refer to a combination of at least two ingredients. In some embodiments, at least one ingredient may be an active agent or otherwise have properties that exert physiologic activity when administered to a subject. For example, amniotic fluid includes at least two ingredients (e.g. water and electrolytes) and is itself a composition or formulation.

As used herein, a “subject” refers to an animal. In one aspect the animal may be a mammal. In another aspect, the mammal may be a human.

As used herein, the terms “release” and “release rate” are used interchangeably to refer to the discharge or liberation of a substance, including without limitation a drug, from the dosage form into a surrounding environment such as an aqueous medium either in vitro or in vivo.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “at least one of” is intended to be synonymous with “one or more of.” For example, “at least one of A, B and C” explicitly includes only A, only B, only C, and combinations of each.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

### Compositions and Methods for Treatment of Endothelial Dysfunction

Many vascular diseases result from oxidative stress that targets the endothelium, which can lead to inflammatory and immune responses that can result in endothelial activation and dysfunction. One of the major consequences of endothelial dysfunction (ED) is an impaired biosynthesis of nitric oxide (NO). NO is a vascular signaling molecule that is involved in the homeostasis of the vascular system. NO is also a vasodilator that maintains response to blood flow in blood vessels. NO can also reduce oxidation of low-density lipoprotein (LDL) to reduce platelet reactivity and decrease leukocyte stickiness in order to protect the vasculature. Consequently, defects in NO production and release due to endothelial dysfunction can induce vasoconstriction, pro-coagulability, and arterial stiffness.

Endothelial cells include the enzyme endothelial nitric oxide synthase (eNOS) that produces NO from amino acid L-arginine in response to blood flow via the endogenous pathway. The EGX is the mechanosensor and signal transducer of blood flow shear stress for eNOS. ED (and a reduction in NO) are implicated in numerous diseases and conditions. Aging can also lead to impaired endothelial function and a reduction of NO biosynthesis in the endothelium. Therefore, effective therapies are needed to address this issue.

The human body constantly generates reactive oxygen species (ROS) via various enzyme systems including mitochondrial enzymes, xanthin oxidase, lipoxygenase, and NADPH oxidase in the endothelium, smooth muscle cells, and adventitia of blood vessels. One of the most damaging ROS of biological significance is superoxide anion (O₂⁻) because of its capacity to generate other more reactive species. The superoxide anion can cause severe oxidative damage inside the body. In one example, the superoxide anion can react with NO to form peroxynitrite (ONOO⁻).

O₂⁻+NO→ONO⁻₂

Peroxynitrite is a powerful oxidant that can damage a wide variety of molecules in cells, including lipids, DNA, and proteins. In fact, this is one of the mechanisms that the immune system uses to kill invading microorganisms. For example, activated macrophages produce NO as an innate immune response to invading pathogens.

Oxidative stress and endothelial dysfunction can induce uncoupling of eNOS, resulting in the generation of O₂⁻ instead of NO. In fact, superoxide itself can directly uncouple eNOS to produce more superoxide, a vicious cycle with devastating consequence to NO production and endothelial function. Therefore, the presence of excess NO and O₂⁻ at the same place and the same time is detrimental to vascular health. Biomarkers such as saliva NO₂ and nitrate (NO₃), breath NO and H₂S, blood antioxidant markers, and also endothelial function can be used to monitor performance and EGX function.

On the other hand, the vascular tissues have antioxidant systems that continuously operate to counterbalance the ROS generated. Some of these antioxidant systems include superoxide dismutase (SOD), catalase, thioredoxins (TRX) peroxidase, glutathione (GSH) peroxidase, and heme oxygenase. Superoxide dismutase is an important antioxidant enzyme that protects from oxidative damage by superoxide radicals. Specifically, SOD converts superoxide anions to hydrogen peroxide (H₂O₂) and oxygen. Hydrogen peroxide can be further reduced to water by other antioxidant enzymes (e.g., catalase and glutathione peroxidase).

2O₂⁻+2H⁺⇄H₂O₂+O₂

Catalase and glutathione peroxidase can stop hydrogen peroxide from reacting with ferrous ion (Fe²⁺) to produce the hydroxyl radical.

In addition to the endogenous pathway, NO can also be derived from dietary nitrite and nitrate. A significant portion of systemic nitric oxide may be generated by reduction of nitrate to nitrite to nitric oxide by other enzymatic systems including the one that exists in the commensal gram-negative bacteria on the tongue.

Because of the prevalence of ED and related diseases and condition, NO can play a role in maintaining endothelial health. In some examples, the endogenous pathway can be aided by supplementation with L-arginine, L-arginine alpha-ketoglutarate, and L-citrulline. However, this approach might not be very effective or efficient in boosting NO biosynthesis. In North America, dietary intake of L-arginine is not a limiting factor for NO synthesis. The human body cannot maintain storage of excess amino acids; rather, the human body either uses them to synthesize proteins or break them down to produce energy. Also, the eNOS enzyme can be reduced in quantity and/or activity with ED. Therefore, supplementation of substrates for eNOS via the endogenous pathway is not adequate.

In other examples, the exogenous pathway can be aided by using inorganic nitrite and nitrate to increase systemic NO. Targeting the exogenous pathway can be effective with a healthy oral microflora. However, oral supplementation of inorganic nitrite and nitrate increases the overall supply of NO in the circulation indiscriminately. This is in contrast to the endogenous pathway that produces NO at a specific location and time in response to blood flow. Since O₂⁻ is also increased with ED, excess NO can react with O₂⁻ to generate peroxynitrite, and result in an increase in oxidative stress. Therefore, supplementation via the exogenous pathway is not adequate either.

The present disclosure describes a number of compositions, dosage forms, and methods that can be used to increase vascular nitric oxide to treat endothelial dysfunction (ED) and related conditions. In some examples, the compositions, dosage forms, and methods described herein can be used to reduce oxidative stress and also restore the EGX.

As a further note, in the present disclosure, it is noted that when discussing the compositions, the dosage forms, and the methods, each of these discussions can be considered applicable to each of these examples, whether or not they are explicitly discussed in the context of that example. Thus, for example, in discussing details about the compositions per se, such discussion also refers to the dosage forms and the methods described herein, and vice versa.

This disclosure includes various ways of boosting NO by using the related activity between NO and hydrogen sulfide (H₂S)—two gasro-transmitters that regulate each other to control vascular tone. While NO is undoubtedly the primary mediator in controlling vascular tone in health and disease, H₂S work together with NO to regulate homeostasis of endothelial function via its vasorelaxing effects. H₂S increases, potentiates, restores, spares, complements, and backs up NO production. It plays a compensatory role for the lack of NO production in the endothelial dysfunction. For example, hydrogen sulfide precursors N-acetyl cysteine (NAC) and S-allyl cysteine (SAC) are antioxidants also known to increase hydrogen sulfide production inside the body via different mechanisms. One way that H₂S interacts with NO and some NO precursors/donors is to generate S-nitrosothiols (SNOS). The small SNOS including S-nitrosocysteine and S-nitrosoglutathione can also transfer NO to other thiol containing molecules such as albumin and hemoglobin via transnitrosation to form S-nitrosoalbumin and S-nitrosohemoglobin. SNOS are widely present in the vascular system with important biological functions including storage, transport and release of NO. Therefore, blood and saliva SNOS levels can be as important biomarkers as those of NO in assessing the NO status and endothelial function.

This disclosure also presents synergistic interactions between the endogenous and exogenous pathways, and various ways of boosting antioxidation to prevent reactions between NO and O₂⁻.

This disclosure further includes ways of restoring endothelial function to enhance NO synthesis via eNOS by repairing, regenerating, and restoring the endothelial glycocalyx (EGX). The EGX is a micro-thin gel layer that coats the entire luminal surface of blood vessels, protects the endothelium from being damaged, and serves as a biomechanical sensor and signal transducer in blood vessels. The EGX can sense the shear stress of blood flow and send the signal to eNOS in the endothelial cell to produce NO. A healthy EGX can generate NO for various physiological functions. The EGX can also mediate and determine various endothelial functions. For example, the EGX harbors many antioxidants including extracellular SOD (ecSOD) that prevents superoxide to damage endothelium and reduce NO availability. Some factors that damage the endothelium can also damage the EGX. Endothelial function can be enhanced by restoring the EGX which can result in further synthesis of NO.

In some cases, O₂⁻can be removed by using specific antioxidant enzymes, such as superoxide dismutase, catalase, or GSH peroxidase (which can target the downstream metabolite H₂O₂ of superoxide dismutase). The EGX can also bind and harbor a variety of soluble molecules derived from both the endothelium and circulating blood that relate to the structural and functional properties of EGX. For example, extracellular superoxide dismutase (ecSOD) can contribute to the antioxidant activities of the EGX to protect the EGX damage.

In other case, O₂⁻ can be removed by using specific nonenzymatic antioxidants including but not limited to glutathione (GSH), vitamin C, and polyphenols/flavonoids. These antioxidants plus lipoic acid, beta carotene, vitamin E, and ubiquinone are also effective against downstream ROS generated from superoxide including hydrogen peroxide, hydroxyl radical, peroxynitrite, and lipid peroxides.

In further detail, a therapeutic composition for treating endothelial dysfunction can include a combination of a nitric oxide (NO) precursor and a hydrogen sulfide (H₂S) precursor in an amount sufficient to sustain an increase of bioavailable NO for the treatment of endothelial dysfunction, and a pharmaceutically acceptable carrier.

The NO precursor can be drawn from various sources, including the exogenous pathway or the endogenous pathway. When the NO precursor is drawn from the endogenous pathway, the NO precursor can include L-arginine, L-arginine alpha-ketoglutarate, L-citrulline, or combinations thereof.

When the NO precursor is drawn from the exogenous pathway, the NO precursor can include inorganic nitrite, inorganic nitrate, organic nitrate, or combinations thereof. In one example, the NO precursor can include nitrite or nitrate salts of sodium, potassium calcium, magnesium, manganese, iron, copper, chromium, zinc, the like, or combinations thereof. In another example, the NO precursor, inorganic nitrite/nitrate, can be derived from arugula, celery, cress, lettuce, chervil, beetroot, spinach, mustard greens, cabbage, fennel, leek, parsley, rocket, swiss chard, leafy chicory, kohlrabi, radish, the like, or combinations thereof, by extraction, concentration, purification, and fermentation technologies. In another example, the NO precursor, inorganic nitrite/nitrate, can be derived from danshen root (Radix salvia miltorrhizae), snakegourd fruit (Fructus trichosanthis), longstamen onion bulb (Bulbus allii macrostemi), sanchi (Radix notoginseng), ginseng (Radix ginseng), borneol (Borneolum syntheticum), and borneol (Cinnamomum), or combinations thereof, by extraction, concentration, purification, and fermentation technologies. Yet in another example, the NO precursor can be an organic nitrate such as thiamine mononitrate. When the NO precursor is drawn from the exogenous pathway, the NO precursor can take various forms. In one example, the NO precursor can be a blend of powdered extracts. In another example, the NO precursor can be a blend of liquid extracts.

In some examples, the NO precursor can be present in the therapeutic composition in a therapeutically effective amount. In some specific examples, the NO precursor can be present in the composition in an amount of from about 0.0001 wt % to about 80 wt %. In another example, the NO precursor can be present in the composition in an amount of from about 0.0001 wt % to about 40 wt %. In another example, the NO precursor can be present in the composition in an amount of from about 0.0001 wt % to about 10 wt %. In another example, the NO precursor can be present in the composition in an amount of from about 0.0001 wt % to about 10 wt %. As a non-limiting example, inorganic nitrate of at least 30 mg, and in some cases 300-450 mg, can be effective. Similarly, for nitrite amounts of at least 3 mg can be effective.

The H₂S precursor can be drawn from various sources as well. In one example, the hydrogen sulfide precursor and stimulator can include sodium hydrosulfide (NaHS), sodium sulfide, N-acetyl cysteine (NAC), S-allyl cysteine (SAC), GSH, thiamine mononitrate, a garlic-derived organic polysulfide, a natural isothiocyanate from in the Brassicacease family such as erucin from arugula and sulforaphane from cruciferous vegetables, a sulfated oligosaccharide, a sulfated polysaccharide, any synthetic sulfur donor, the like, other organosulfur compounds (OSCs), synthetic H₂S donors such as phosphorodithioate derivatives, NOSH compounds which act as both NO and H₂S precursors, or combinations thereof.

In another example, the hydrogen sulfide precursor can be selected from a group consisting of: sodium hydrosulfide (NaHS), N-acetyl cysteine (NAC), S-allyl cysteine (SAC), GSH, thiamine mononitrate, a garlic-derived organic polysulfide, a natural isothiocyanate from in the Brassicacease family such as erucin from arugula and sulforaphane from cruciferous vegetables, any synthetic sulfur donor, the like, or combinations thereof. In one example, the H₂S precursor can be substantially free of a sulfated oligosaccharide or a sulfated polysaccharide.

In some examples, the H₂S precursor can be present in the therapeutic composition in a therapeutically effective amount. In some specific examples, the H₂S precursor can be present in the composition in an amount of from about 0.0001 wt % to about 80 wt %. In another example, the H₂S precursor can be present in the composition in an amount of from about 0.0001 wt % to about 90 wt %. In another example, the H₂S precursor can be present in the composition in an amount of from about 0.0001 wt % to about 60 wt %. In another example, the H₂S precursor can be present in the composition in an amount of from about 0.0001 wt % to about 30 wt %.

In some further examples, the combination of the NO precursor and the H₂S precursor can be present in an amount sufficient to treat endothelial dysfunction. In one example, the NO precursor and the H₂S precursor can be present in amounts such that the ratio of amount (wt %) of NO precursor to amount (wt %) of H₂S precursor is about 1:1. In another example, the NO precursor and the H₂S precursor can be present in amounts such that the ratio of amount (wt %) of NO precursor to amount (wt %) of H₂S precursor is from about 1:1 to about 1:10. In another example, the NO precursor and the H₂S precursor can be present in amounts such that the ratio of amount (wt %) of NO precursor to amount (wt %) of H₂S precursor is from about 10:1 to about 1:1.

In some further examples, the therapeutic composition can also include an antioxidant. In one example, the antioxidant can include superoxide dismutase (SOD), catalase, glutathione peroxidase, the like, or combinations thereof.

In some cases, the antioxidant can help inhibit oxidation of the nitric oxide precursor or hydrogen sulfide precursor or other ingredients in the therapeutic composition. In other cases, the antioxidant can facilitate the reduction of nitrite to nitric oxide in the body. In some examples, the antioxidant can provide a therapeutic effect when administered in connection with the nitric oxide precursor or hydrogen sulfide precursor.

A variety of antioxidants can be used in the therapeutic composition. In some examples, the antioxidant can include butylated hydroxyanisole (BHA), butylated hydroxytoluene (BHT), ascorbic acid, ascorbyl palmitate, alpha tocopherol, potassium metabisulfite, sodium thiosulfate, propyl gallate, carotenoids, lipoic acid, ubiquinone, the like, or a combination thereof. In other examples, the antioxidant can include a plant-based powder blend rich in antioxidants such as polyphenols. In the case of cardiovascular disorders, oxidative stress and reactive oxygen species (ROS) can cause endothelial damage, progression of atherosclerosis, injury in sustained myocardial infarction and/or in ischemia reperfusion, the like, or a combination thereof. A deterioration in nitric oxide (NO) dependent vasorelaxation is a risk factor that can predispose individuals to cardiovascular disease.

Plant-based antioxidants can also be used in the therapeutic composition. In some cases, the antioxidants can be derived from red grape skin extract, red grape seed extract, white grape skin extract, white grape seed extract, green tea extract, carrot juice or extract, tomato juice or extract, broccoli juice or extract, green cabbage juice or extract, onion juice or extract, garlic juice or extract, asparagus juice or extract, olive juice or extract, cucumber juice or extract, bilberry juice or extract, grapefruit juice or extract, papaya juice or extract, pineapple juice or extract, strawberry juice or extract, apple juice or extract, apricot juice or extract, cherry juice or extract, orange juice or extract, black currant juice or extract, beetroot, (tartar) cherry, kiwi fruit, watermelon, hawthorn berry, celery, cili fruit, jujube fruit, broccoli, blue honeysuckle fruit, strawberry, yumberry, purple sweet potato, monk fruit, plum, and the like, or a combination thereof.

The antioxidant can be present in the therapeutic composition in a variety of amounts. In some examples, the antioxidant can be present in the therapeutic composition in an amount sufficient to contribute to the antioxidant activity of the endothelial glycocalyx (EGX). In some examples, the antioxidant can be present in the therapeutic composition in a therapeutically effective amount. In some specific examples, the antioxidant can be present in the composition in an amount of from about 0.0001 wt % to about 90 wt %. In another example, the antioxidant can be present in the composition in an amount of from about 0.0001 wt % to about 30 wt %. In another example, the antioxidant can be present in the composition in an amount of from about 0.0001 wt % to about 10 wt %. In some cases, the composition can be free of antioxidant.

In some further examples, the therapeutic composition can also include an endothelial glycocalyx regenerator. In one example, the glycocalyx regenerator can include a sulfated polysaccharide, a sulfated oligosaccharide, chito-oligosaccharides (COS), other glycans and their precursors such as glucosamine, hyaluronan and chondroitin sulfate, or combinations thereof. In another example, the glycocalyx regenerator can include rhamnan sulfate, fucoidan sulfate, carrageenan, or combinations thereof.

In another example, the glycocalyx regenerator can be present in the composition in an amount of from about 0.0001 wt % to about 80 wt %. In another example, the glycocalyx regenerator can be present in the composition in an amount of from about 0.0001 wt % to about 30 wt %. In another example, the glycocalyx regenerator can be present in the composition in an amount of from about 0.0001 wt % to about 20 wt %. In another example, the glycocalyx regenerator can be present in the composition in an amount of from about 0.0001 wt % to about 10 wt %. In some cases, the composition can be free of glycocalyx regenerator.

In some other examples, the therapeutic composition can also include a source of nitrate and/or nitrite. Dietary nitrate and nitrite are precursors of nitric oxide (NO) that plays important roles in maintain endothelial function. In addition to the endothelial nitric oxide synthase, a significant portion of systemic nitric oxide may be generated by reduction of nitrate to nitrite to nitric oxide by other enzymatic systems including the one that exists in the commensal gram-negative bacteria on the tongue. Nitric oxide is a vasodilator that increases blood flow. It also has anti-inflammatory, anticoagulant and antioxidant activities in relation to the development of atherosclerosis. It has been shown that dietary nitrate and nitrite lower blood pressure in humans.

The therapeutic composition can include a variety of source of nitrate and nitrite. Non-limiting examples include thiamine mononitrate and nitrate and nitrite salts of sodium, potassium, calcium, magnesium, manganese, iron, copper, chromium and zinc. Also many fruits and vegetables are good sources of nitrate and nitrite. A non-exhausting list includes arugula, celery, cress, lettuce, chervil, beetroot, spinach, mustard greens, cabbage, fennel, leek, parsley, rocket, swiss chard, leafy chicory, kohlrabi, radish, etc. Many herbs such as traditional Chinese medicinal herbs also contain an appreciable amount of nitrate and nitrite. They include, but are not limited to, danshen root (Radix salvia miltorrhizae), snakegourd fruit (Fructus trichosanthis), longstamen onion bulb (Bulbus allii macrostemi), sanchi (Radix notoginseng), ginseng (Radix ginseng), borneol (Borneolum syntheticum), and borneol (Cinnamomum). In some specific examples, the plant-based nitrate and nitrite can include a blend of powdered extracts. In still other examples, the plant-based nitrate and nitrite can include a blend of liquid extracts or juices.

In some examples, the nitrate and nitrite can be present in the therapeutic composition in an amount from about 10 wt % to about 90 wt %. In other examples, the nitrate and nitrite can be present in an amount from about 20 wt % to about 80 wt %.

As non-limiting examples, the therapeutic composition can include an NO precursor, an H₂S precursor, and pharmaceutically acceptable carrier with one or more supplemental components such as an antioxidant, a glycocalyx regenerator, other cofactors supporting both endogenous and exogeneous NO pathways, and the like. When provided as a powdered supplement optional additives can be added such as, but not limited to, sweeteners, colorants, flavorants, preservatives, and the like.

Furthermore, the therapeutic composition can also include one or more factors that optimize the eNOS function and activity. Non-limiting examples of suitable factors can include at least one of magnesium, zinc, pyridoxine (B6), folic acid (B9), vitamin B12, vitamin C, vitamin D, and tetrahydrobiopterin (BH4).

The therapeutic composition can also include a pharmaceutically acceptable carrier. The nature of the pharmaceutically acceptable carrier can depend on the intended mode of administration. In some examples, the therapeutic composition can be formulated for administration via injection. In other examples, the therapeutic composition can be formulated for oral administration. In yet another example, the therapeutic composition can be formulated for IV administration.

Where the therapeutic composition is formulated for administration via injection, the pharmaceutically acceptable carrier can include one or more components suitable for such a composition. Non-limiting examples can include water, a solubilizing or dispersing agent, a tonicity agent, a pH adjuster or buffering agent, a preservative, a chelating agent, a bulking agent, the like, or a combination thereof.

In some examples, an injectable therapeutic composition can include a solubilizing or dispersing agent. Non-limiting examples of solubilizing or dispersing agents can include polyoxyethylene sorbitan monooleates, lecithin, polyoxyethylene polyoxypropylene co-polymers, propylene glycol, glycerin, ethanol, polyethylene glycols, sorbitol, dimethylacetamide, polyethoxylated castor oils, n-lactamide, cyclodextrins, caboxymethyl cellulose, acacia, gelatin, methyl cellulose, polyvinyl pyrrolidone, the like, or combinations thereof.

In some examples, an injectable therapeutic composition can include a tonicity agent. Non-limiting examples of tonicity agents can include sodium chloride, potassium chloride, calcium chloride, magnesium chloride, mannitol, sorbitol, dextrose, glycerin, propylene glycol, ethanol, trehalose, phosphate-buffered saline (PBS), Dulbecco's PBS, Alsever's solution, Tris-buffered saline (TBS), water, balanced salt solutions (BSS), such as Hank's BSS, Earle's BSS, Grey's BSS, Puck's BSS, Simm's BSS, Tyrode's BSS, and BSS Plus, the like, or combinations thereof. The tonicity agent can be used to provide an appropriate tonicity of the therapeutic composition. In one aspect, the tonicity of the therapeutic composition can be from about 250 to about 350 milliosmoles/liter (mOsm/L). In another aspect, the tonicity of the therapeutic composition can be from about 277 to about 310 mOsm/L.

In some examples, an injectable therapeutic composition can include a pH adjuster or buffering agent. Non-limiting examples of pH adjusters or buffering agents can include a number of acids, bases, and combinations thereof, such as hydrochloric acid, phosphoric acid, citric acid, sodium hydroxide, potassium hydroxide, calcium hydroxide, acetate buffers, citrate buffers, tartrate buffers, phosphate buffers, triethanolamine (TRIS) buffers, the like, or combinations thereof. Typically, the pH of the therapeutic composition can be from about 5 to about 9, or from about 6 to about 8.

In some examples, an injectable therapeutic composition can include a preservative. Non-limiting examples of preservatives can include ascorbic acid, acetylcysteine, bisulfite, metabisulfite, monothioglycerol, phenol, meta-cresol, benzyl alcohol, methyl paraben, propyl paraben, butyl paraben, benzalkonium chloride, benzethonium chloride, butylated hydroxyl toluene, myristyl gamma-picolimium chloride, 2-phenoxyethanol, phenyl mercuric nitrate, chlorobutanol, thimerosal, tocopherols, the like, or combinations thereof.

In some examples, an injectable therapeutic composition can include a chelating agent. Non-limiting examples of chelating agents can include ethylenediaminetetra acetic acid, calcium, calcium disodium, versetamide, calteridol, diethylenetriaminepenta acetic acid, the like, or combinations thereof.

In some examples, an injectable therapeutic composition can include a bulking agent. Non-limiting examples of bulking agents can include sucrose, lactose, trehalose, mannitol, sorbitol, glucose, rafinose, glycine, histidine, polyvinyl pyrrolidone, the like, or combinations thereof.

Where the therapeutic composition is formulated for oral administration, the pharmaceutically acceptable carrier can include one or more components suitable for such a composition. In the case of solid oral compositions or dosage forms, the pharmaceutically acceptable carrier can include a variety of components suitable for forming a capsule, tablet, or the like. In the case of a liquid oral composition or dosage form, the pharmaceutically acceptable carrier can include a variety of components suitable for forming a dispersion, a suspension, a syrup, an elixir, or the like.

In some specific examples, the therapeutic composition can be formulated as a tablet. In such examples, the therapeutic composition can typically include a binder. Non-limiting examples of binders can include lactose, calcium phosphate, sucrose, corn starch, microcrystalline cellulose, gelatin, polyethylene glycol (PEG), polyvinyl pyrrolidone (PVP), hydroxypropyl cellulose, hydroxyethylcellulose, carboxymethyl cellulose (CMC), cellulose, other cellulose derivatives, the like, or combinations thereof.

Where the therapeutic composition is formulated as a tablet, in some examples the therapeutic composition can also include a disintegrant. Non-limiting examples of disintegrants can include crosslinked PVP, crosslinked CMC, modified starch, sodium starch glycolate, the like, or combinations thereof. In some examples, the tablet can also include a filler. Non-limiting examples of fillers can include lactose, dicalcium phosphate, sucrose, microcrystalline cellulose, the like, or combinations thereof. In some further examples, the tablet can include a coating. Such coatings can be formed with a variety of materials, such as hydroxypropyl methylcellulose (HPMC), shellac, zein, various polysaccharides, various enterics, the like, or combinations thereof.

In some examples, the tablet can include a variety of other ingredients, such as anti-adherents (e.g. magnesium stearate, calcium stearate, for example), colorants (e.g. titanium dioxide, carmine, for example), glidants (e.g. fumed silica, talc, magnesium carbonate, for example), lubricants or anti-caking agents (e.g. talc, silicon dioxide, magnesium stearate, calcium stearate, stearic acid, for example) preservatives, desiccants, and/or other suitable tablet excipients, as desired.

In some other examples, the therapeutic composition can be formulated as a capsule. In such examples, the capsule itself can typically include gelatin, hypromellose, HPMC, CMC, other plant-based capsule materials, the like, or combinations thereof. A variety of excipients can also be included within the capsule, such as binders, disintegrants, fillers, glidants, anti-caking agents, preservatives, coatings, the like, or combinations thereof, such as those listed above with respect to tablets, for example, or other suitable variations.

In some examples, the therapeutic composition can be formulated as a liquid therapeutic composition or liquid oral dosage form. A liquid oral dosage form can include a variety of excipients, such as a liquid vehicle, a solubilizing agent, a thickener or dispersant, a preservative, a tonicity agent, a pH adjuster or buffering agent, a sweetener, a thickening agent, the like, or a combination thereof. Non-limiting examples of liquid vehicles can include water, ethanol, glycerol, propylene glycol, the like, or combinations thereof. Non-limiting examples of solubilizing agents can include banzalkonium chloride, benzethonium chloride, cetylpyridinium chloride, docusate sodium, nonoxynol-9, octoxynol, polyoxyethylene polyoxypropylene co-polymers, polyoxyl castor oils, polyoxyl hydrogenated castor oils, polyoxyl oleyl ethers, polyoxyl cetylstearyl ethers, polyoxyl stearates, polysorbates, sodium lauryl sulfate, sorbitan monolaurate, sorbitan monooleate, sorbitan monopalmitate, sorbitan monostearate, tyloxapol, the like, or combinations thereof. Non-limiting examples of thickeners or dispersants can include sodium alginate, methylcellulose, hydroxyethylcellulose, hydroxypropylcellulose, HPMC, CMC, microcrystalline cellulose, tragacanth, xanthan gum, bentonite, carrageenan, guar gum, colloidal silicon dioxide, the like, or combinations thereof. The preservative, tonicity agent, pH adjuster or buffering agent can typically be any of those described above with respect to the injectable formulations or other suitable preservative, tonicity agent, pH adjuster or buffering agent. Sweeteners can include natural and/or artificial sweeteners, such as sucrose, glucose, fructose, stevia, erythritol, xylitol, aspartame, sucralose, neotame, acesulfame potassium, saccharin, advantame, sorbitol, the like, or combinations thereof, for example.

In some examples, the therapeutic composition can be formulated as a functional food and/or medical food product such as a food bar, powder, or beverage. Food bars can be formulated to fit different dietary regiments for any specific purposes such as weight loss, energy, meal replacement, high protein, high fiber, low glycemic, etc. A food bar usually contains ingredients that supply energy-yielding nutrients such as carbohydrate, protein and lipid as well as other macro- and micronutrients including but not limited to vitamins and minerals. Other health promoting ingredients such fruit and vegetable powder may be included in the formulation in addition to filler, binder, emulsifier, water, humectant, flavor, color, sweetener, preservative, etc. The therapeutic composition can be formulated into a food bar with other ingredients to achieve desirable health benefits, taste, texture, flavor and stability. Similarly, the therapeutic composition may be formulated into a powder such as a protein powder, meal replacement powder, or functional beverage dry mix. It can also be formulated into a functional drink. A ready to drink beverage may contain other ingredients including various nutrients, health promoting agents, pH adjustor (acidity regulator), electrolyte, flavor, sweetener, stabilizing agent, color, preservative, etc.

The present disclosure also describes oral dosage forms. The oral dosage forms can include a combination of a nitric oxide (NO) precursor and a hydrogen sulfide (H₂S) precursor in an amount sufficient to treat endothelial dysfunction. The oral dosage form can also include a pharmaceutically acceptable carrier.

The types of NO precursors or hydrogen sulfide precursors that can be included in the oral dosage forms are generally described above with respect to the therapeutic compositions. In some examples, the oral dosage form can include NO precursors or hydrogen sulfide precursors in an amount from about 0.5 mg to about 5,000 mg of NO precursors, hydrogen sulfide precursors, or both. In some other examples, the oral dosage form can include from about 5 mg to about 1500 mg of NO precursors, or hydrogen sulfide precursors, or both per dose. In some additional examples, the oral dosage form can include from about 10 mg to about 800 mg of NO precursors, or hydrogen sulfide precursors, or both per dose. In still other examples, the oral dosage form can include from about 50 mg to about 300 mg of NO precursors, or hydrogen sulfide precursors, or both per dose.

In some additional examples, the oral dosage forms can include an antioxidant, either antioxidant enzymes and/or nonenzymatic antioxidants, as described above with respect to the therapeutic compositions. In some examples, the oral dosage form can include the plant antioxidant in an amount from about 20 mg to about 20,000 mg per dose. In other examples, the antioxidant can be present in the oral dosage form in an amount from about 100 mg to about 600 mg per dose. In still other examples, the antioxidant can be present in the oral dosage form in an amount from about 125 mg to about 350 mg per dose. In some specific examples, the antioxidant can be included in the oral dosage form in the form of a powdered blend of edible plant materials with antioxidant activity, such as those described above. In other examples, the antioxidant can be included in the oral dosage form in the form of a liquid blend of edible plant materials with antioxidant activity, such as those described above.

In some additional examples, the oral dosage forms can include a source of nitrate and nitrite, as described above with respect to the therapeutic compositions. In some examples, the oral dosage form can include nitrate and nitrite in an amount from about 20 mg to about 2,000 mg per dose. In other examples, nitrate and nitrite can be present in the oral dosage form in an amount from about 50 mg to about 1,000 mg per dose. In still other examples, nitrate and nitrite can be present in the oral dosage form in an amount from about 200 mg to about 600 mg per dose. In some specific examples, nitrate and nitrite can be included in the oral dosage form in the form of a powdered blend of edible plant materials with nitrate and nitrite, such as those described above. In other examples, nitrate and nitrite can be included in the oral dosage form in the form of a liquid blend of edible plant materials with nitrate and nitrite, such as those described above.

In some specific examples, the oral dosage forms can be solid oral dosage forms. Where this is the case, the solid oral dosage forms can include any pharmaceutically acceptable carrier components suitable for a solid oral dosage form. In some specific examples, the solid oral dosage form can include one or more of a binder, a disintegrant, a filler, an anti-adherent, a colorant, a glidant, a lubricant or anti-caking agent, a preservative, a desiccant, the like, or a combination thereof, such as those described above with respect to the therapeutic compositions. In some examples, the solid oral dosage form can be formulated as a tablet. In other examples, the solid oral dosage form can be formulated as a two-piece hard capsule or a hermetically sealed soft-gel capsule.

In some additional specific examples, the oral dosage forms can be liquid oral dosage forms. Where this is the case, the liquid oral dosage forms can include any pharmaceutically acceptable carrier components suitable for a liquid oral dosage form. In some specific examples, the liquid oral dosage form can include a liquid vehicle, a solubilizing agent, a thickener or dispersant, a preservative, a tonicity agent, a pH adjuster or buffering agent, a sweetener, the like, or a combination thereof, such as those described above.

In some examples, the dosage forms or therapeutic compositions described herein can be disposed in a suitable container. Such containers can include multiple-use containers or single use containers. Non-limiting examples can include bottles, vials, blister packs, bags, or the like. In some examples, the container can be an amber colored container or other suitable container configured to protect the dosage form or therapeutic composition from light. In yet other examples, the container can include instructions and dosing information for the dosage form or therapeutic composition. The container can include a variety of materials, such as polyethylene, polypropylene, polycarbonate, polyvinyl chloride, glass, the like, or a combination thereof.

In yet additional alternatives, the therapeutic compositions described herein can be used as a food additive to fortify a food supply for general population. For example, the therapeutic composition can be safely introduced into a systemic food supply such as, but not limited to, milled grain flours, pastas, breakfast cereals, bread, soup or soup mixes, food bars, spices, condiments, dairy products, beverages, drink mixes, frozen food items, pastries, cookies and crackers, snacks, or the like.

As an example formulation, a therapeutic composition can include two NO precursors, an H₂S precursor, an endothelial glycocalyx regenerating compound, a nonenzymatic antioxidant, a plant-based antioxidant, and one or more eNOS cofactors. In a further example, the two NO precursors can include potassium nitrate (100-500 mg/serving) and beetroot extract (50-2000 mg/serving), the H₂S precursor can include SAC-rich fermented black garlic extract (100-1000 mg/serving) and thiamine mononitrate (10-200 mg/serving), the eGRC includes MNE (25-500 mg/serving), the nonenzymatic antioxidant includes vitamin C (30-300 mg/serving), the plant-based antioxidant includes bilberry extract (50-500 mg/serving), and the eNOS cofactors include magnesium (10-200 mg/serving), zinc (1-30 mg/serving), methyl cobalamin (10-200 mcg/serving), and cholecalciferol (2.5-100 mcg/serving).

The present disclosure also describes a method of treating endothelial dysfunction. The method can include identifying endothelial glycocalyx (EGX) and/or endothelial dysfunction of the subject and administering to the subject a combination of a nitric oxide (NO) precursor and a hydrogen sulfide (H₂S) precursor in an amount and at a frequency sufficient to stabilize and restore the EGX and endothelial function. In one aspect, damage in the EGX and endothelium can be detected using various blood markers, imaging technologies, and functional tests. For example, biomarkers, Alpha Elution Technology, dark field microscopy, coronary epicardial vasoactivity, flow-mediated dilation, plethysmography, and EndoPat may be used to assess EGX or endothelial dysfunction.

Administration of the combination of a nitric oxide (NO) precursor and a hydrogen sulfide (H₂S) precursor to a subject can be performed in a number of ways. In some examples, the combination of a nitric oxide (NO) precursor and a hydrogen sulfide (H₂S) precursor can be administered orally. Oral administration can include administration as a solid oral dosage form (e.g. a tablet, a capsule, etc.) or a liquid oral dosage form (e.g. a solution, a suspension, a syrup, an elixir, a gel, etc.). In some other examples, administration can be performed via injection (e.g. intravenous, intra-arterial, intramuscular, sub-cutaneous, etc.). Further, where the composition is administered via injection, it can be injected via a bolus injection or via metered infusion. Other forms of administration can also include topical administration, transdermal administration, inhalation, ophthalmic administration, nasal administration, otic administration, administration as a suppository, or the like.

The particular therapeutic composition administered can be any of those described herein, or the like. Further, in some examples, the combination of a nitric oxide (NO) precursor and a hydrogen sulfide (H₂S) precursor can be administered as a composition or dosage form, such as those described herein. In some examples, the combination of the NO precursor and the H₂S precursor can be administered in an amount from about 1 mg to about 5,000 mg per dose. In some other examples, the oral dosage form can be administered in an amount from about 10 mg to about 1500 mg of the combination of the NO precursor and the H₂S precursor per dose. In some additional examples, the oral dosage form can be administered in an amount from about 30 mg to about 300 mg of the combination of the NO precursor and the H₂S precursor per dose. In still other examples, the oral dosage form can be administered in an amount from about 50 mg to about 200 mg of the combination of the NO precursor and the H₂S precursor per dose. It is also noted that where the combination of the NO precursor and the H₂S precursor is administered as part of a solid oral dosage form, a dose can include one, two, three, four, or more capsules, tablets, etc.

The combination of the NO precursor and the H₂S precursor can be administered at a variety of frequencies. In some examples, a dose of the combination of the NO precursor and the H₂S precursor can be administered at a frequency of from once daily to four times daily. In some examples, a dose of the combination of the NO precursor and the H₂S precursor can be administered once per day, twice per day, three times per day, four times per day, or more. In other examples, the combination of the NO precursor and the H₂S precursor can be administered at a frequency of from about once every two days, three days, five days, or seven days, for example. Thus, a variety of suitable administration frequencies can be employed with the present methods.

Further, administration can continue for a variety of durations, depending on the desired treatment outcome. In some examples, administration can continue while symptoms of ED persist. In other examples, administration can be ongoing as either a prophylactic or intervention treatment. In still other examples, administration can continue until the ED has resolved or a threshold level of NO has been reached. Other suitable durations of administration can also be employed, as desired. As a general guideline, administration duration can be from about 2 weeks to about 24 months, and often from 2 months to 12 months.

In some examples, the combination of the NO precursor and the H₂S precursor can be administered in connection (e.g. co-administered) with another active agent. In some examples, the second active agent can include an antioxidant, nitrate, nitrite, or a combination thereof, as described elsewhere herein.

The present method takes advantage of synergistic mechanisms to generate and store NO which sustains a therapeutic level of NO in the vascular system beyond what can achieved by the existing approaches. Thus, the amount and frequency of therapeutic composition can be adjusted to achieve an increase of bioavailable NO for treatment of ED. In some cases, such an increase can be sustained for a period of at least 12 hours, 24 hours, or 48 hours. In many examples, the increase can be sustained for at least 24 hours such that daily administration can be effective for longer term treatment and recovery.

It is also noted that the present methods can be used to treat a number of adverse health conditions related to endothelial dysfunction. Non-limiting examples of adverse health conditions can include coronary heart disease, myocardial infarction, carotid artery disease, stroke, peripheral artery disease, aneurysms, chronic kidney disease, erectile dysfunction, hypertension, Alzheimer's disease, vascular dementia, diabetes, Raynaud's disease, sleep apnea, the like, or a combination thereof.

### EXAMPLES

The following examples are provided to promote a clearer understanding of certain embodiments of the present invention and are in no way meant as a limitation thereon. All statistical analysis was performed using two-tailed and paired Student's t-test to compare two means.

### Example 1—A Basic NO Precursor Composition (Formula 1)

A basic NO precursor containing composition was prepared by using the components set forth in Table I. The composition was prepared by weighing all of the components into a clean stainless-steel container and combining in a substantially inert environment to form a solution. A predetermined quantity of the fill material was disposed into a hard capsule to get the target NO precursor dose per dosage unit. The capsules were allowed to cool at room temperature, banded (if required) and packaged in a HDPE bottle and tightly closed with an appropriate lid.

TABLE I
(Formula 1)

| Function | Ingredient | Amount (mg/serving) | |
|---|---|---|---|
| NO Precursor | Potassium Nitrate | 500 | mg |
| Antioxidant and reducing | Ascorbic Acid | 300 | mg |
| agent to facilitate NO | (Vitamin C) | | |
| metabolism | | | |
| Organosulfur NO donor | Thiamine Mononitrate | 90 | mg |
| | (Vitamin B1) | | |
| Increase NO and NO- | Magnesium | 75 | mg |
| dependent vasodilation | | | |
| Support eNOS cofactor | Methyl Cobalamin | 200 | mcg |

| | |
|---|---|
| BH4 | (Vitamin B12) |

The composition was evaluated for its ability to increase NO production and availability. A healthy young male ingested a single dose of the composition in the morning only and his saliva nitrite (NO₂) was determined with Berkeley Test® Nitric Oxide Test Strip over 12 hours (Formula 1 in Table II). Saliva nitrite is a surrogate marker of NO bioavailability as saliva conversion of nitrate to nitrite (NO₃) is a step for NO generation via exogenous pathway inside the body. Saliva nitrite has been shown to parallel blood nitrite in published studies. Saliva nitrite test strip results were recorded based on a five-color scale following the manufacturer's instructions, i.e., 20 μM (depleted), 110 μM (low), 220 μM (threshold), 435 μM (target), and 870 μM (high).

TABLE II
(Saliva Nitrite)

| | | Formula 1 | Formula 2 | Formula 3 |
|---|---|---|---|---|
| Hour | Baseline (μM) | (μM) | (μM) | (μM) |
| 0 | 20 | >20 and <110 | 20 | 20 |
| 2 | 20 | >20 and <110 | 110 | 110 |
| 4 | 20 | 110 | >110 and <220 | 220 |
| 6 | 20 | >110 and <220 | 220 | >110 and <220 |
| 8 | >20 and <110 | >20 and <110 | >110 and <220 | 110 |
| 10 | 20 | 110 | 110 | 110 |
| 12 | 20 | >20 and <110 | >20 and <110 | >20 and <110 |

Saliva nitrite at the baseline was very low and relatively flat over 12 hours. After ingesting the composition (Formula 1), saliva nitrite started to increase at 4 hours and reached the peak at 6 hours. After 12 hours, saliva nitrite dropped back to the initial level. It is clear that intake of formula 1 led to a marked increase of NO bioavailability over baseline for at least 12 hours.

### Example 2—A Basic NO and H₂S Precursor Composition (Formula 2)

An H₂S precursor containing composition was prepared by adding N-acetyl cysteine to the composition in Example 1 (Formula 2 in Table III). The composition was prepared by weighing all of the components into a clean stainless-steel container and combining in a substantially inert environment to form a solution. A predetermined quantity of the fill material was disposed into a capsule to get the target H₂S precursor dose per dosage unit. The capsules were allowed to cool at room temperature, banded (if required) and packaged in a HDPE bottle and tightly closed with an appropriate lid.

TABLE III
(Formula 2)

| Function | Ingredient | Amount (mg/serving) | |
|---|---|---|---|
| NO Precursor | Potassium Nitrate | 500 | mg |
| H₂S Precursor | N-acetyl cysteine | 1,200 | mg |
| Antioxidant and reducing | Ascorbic Acid | 300 | mg |
| agent to facilitate NO | (Vitamin C) | | |
| metabolism | | | |
| Organosulfur NO donor | Thiamine Mononitrate | 90 | mg |
| | (Vitamin B1) | | |
| Increase NO and NO- | Magnesium | 75 | mg |
| dependent vasodilation | | | |
| Support eNOS cofactor | Methyl Cobalamin | 200 | mcg |

| | |
|---|---|
| BH4 | (Vitamin B12) |

The composition was evaluated for its ability to increase NO production and availability. A healthy young male ingested a single dose of the composition in the morning only and his salivary nitrite (NO₂) was determined with Berkeley Test® Nitric Oxide Test Strip over 12 hours (Formula 2 in Table II).

Compared to baseline, saliva nitrite after ingesting Formula 2 was higher than the baseline at every time point. In addition, saliva nitrite after ingesting Formula 2 was higher than that after ingesting Formula 1 for 4 of the 7 time points (from 2 hours to 8 hours). It is worth noting the starting saliva nitrite was higher for Formula 1 than the starting concentration of 20 μM for Formula 2. At 12 hours, saliva nitrite dropped back to the initial value for Formula 1. In contrast, saliva nitrite is still higher than the initial value for Formula 2 at the 12-hour mark. Therefore, it is concluded that Formula 2 is superior to Formula 1. It sustains a higher level of bioavailable NO for more than 12 hours.

We hypothesized that formula 2 sustained a higher level of bioavailable NO than formula 1 because N-acetyl cysteine generates hydrogen sulfide in the body. To test this hypothesis, we measured transdermal hydrogen sulfide on the surface of the forearm after an adult male subject ingested 600 mg of N-acetyl cysteine. The test was performed using Interscan Halimeter® PLUS with a simple modification of sample collection method. Hydrogen sulfide, the primary volatile sulfur compounds detectable by the device, was measured in triplicate for 6 hours after dosing with N-acetyl cysteine (Table IV). The transdermal hydrogen sulfide increased within 2 hours from 16 ppm to 27 ppm. It peaked at 51 ppm at 4 hours and returned to the baseline after 6 hours. The data support a mechanism wherein N-acetyl cysteine increases hydrogen sulfide production that boosts NO availability and activity in the body.

TABLE IV
(Transdermal Hydrogen Sulfide)

| | | | | | | |
|---|---|---|---|---|---|---|
| Time (h) | 0 | 2 | 3 | 4 | 5 | 6 |
| H₂S (ppb) | 16 | 27 | 46 | 51 | 40 | 16 |

### Example 3—A NO Precursor with an eGRC (Formula 3)

The NO precursor composition in Example 1 was enhanced by adding an endothelial glycocalyx regenerating compound (eGRC), a green seaweed Monostroma nitidum extract (MNE) as Formula 3 in Table V and a method similar to that described in Examples 1 and 2.

TABLE V
(Formula 3)

| Function | Ingredient | Amount (mg/serving) | |
|---|---|---|---|
| NO Precursor | Potassium Nitrate | 500 | mg |
| eGRC | MNE | 300 | mg |
| Antioxidant and reducing | Ascorbic Acid | 300 | mg |
| agent to facilitate NO | (Vitamin C) | | |
| metabolism | | | |
| Organosulfur NO donor | Thiamine Mononitrate | 90 | mg |
| | (Vitamin B1) | | |
| Increase NO and NO- | Magnesium | 75 | mg |
| dependent vasodilation | | | |
| Support eNOS cofactor | Methyl Cobalamin | 200 | mcg |

| | |
|---|---|
| BH4 | (Vitamin B12) |

The composition was evaluated for its ability to increase NO production and availability. A healthy young male ingested a single dose of the composition in the morning only and his salivary nitrite (NO₂) was determined with Berkeley Test® Nitric Oxide Test Strip over 12 hours (Formula 3 in Table II).

As one can see, all data points of saliva nitrite for Formula 3 were higher than those of baseline. Also 3 of 7 data points (2, 4 and 8 hour) of Formula 3 are higher than those of Formula 1. It is worth noting the starting saliva nitrite was higher for Formula 1 than the starting concentration of 20 μM for Formula 3. At 12 hours, saliva nitrite dropped back to the initial value for Formula 1. In contrast, saliva nitrite was still higher than the initial value for Formula 3 at the 12-hour mark. Therefore, it is concluded that Formula 3 is superior to Formula 1. It sustains a higher level of bioavailable NO for more than 12 hours.

### Example 4—An Advanced NO Precursor with H₂S Precursor Composition (Formula 4)

An advanced composition was prepared by including plant based NO precursor with H₂S precursor (Formula 4 in Table VI). This is a plant-based formula with NO and H₂S precursors derived from plant materials. The plant-based precursors have the advantage of containing other benefiting phytochemicals such as antioxidant polyphenols. They may also have better absorption and release profiles to increase NO bioavailability. Formula 4 also includes additional plant polyphenols and vitamin D₃, both increase eNOS activity and NO bioavailability. This formula actually contains less NO precursor and less H₂S precursor than Formula 3. However, it was expected to work better due to the synergistic effect of its components.

The composition was prepared by weighing all of the components into a clean stainless-steel container and combining in a substantially inert environment to form a solution. The composition was packaged and stored in a container.

TABLE VI
(Formula 4)

| | | Amount | |
|---|---|---|---|
| Function | Ingredient | (mg/serving) | |
| NO Precursor | Potassium Nitrate | 380 | mg |
| NO Precursor and | Beetroot Extract | 200 | mg |
| Antioxidant | | | |
| H₂S Precursor and | Fermented Garlic | 400 | mg |
| Antioxidant | Extract (SAC) | | |
| Antioxidant and reducing | Ascorbic Acid | 180 | mg |
| agent to facilitate NO | (Vitamin C) | | |
| metabolism | | | |
| Plant polyphenols and | Bilberry Extract | 100 | mg |
| regulating NO | | | |
| bioavailability | | | |
| Organosulfur NO donor | Thiamine Mononitrate | 80 | mg |
| | (Vitamin B1) | | |
| Increase NO and NO- | Magnesium | 56 | mg |
| dependent vasodilation | | | |
| Enhance eNOS function | Zinc | 3 | mg |
| Support eNOS cofactor | Methyl Cobalamin | 100 | mcg |
| BH4 | (Vitamin B12) | | |
| Increase/regulate eNOS and | Cholecalciferol | 60 | mcg |

| | |
|---|---|
| SOD expression | (Vitamin D₃) |

The composition was evaluated for its ability to increase NO bioavailability. A healthy young male ingested a single dose of the composition in the morning only. His saliva nitrite was determined with Berkeley Test® Nitric Oxide Test Strip over 12 hours (Formula 4 in Table VII).

TABLE VII
(Saliva Nitrite)

| Hour | Baseline (μM) | Formula 4 (μM) | Formula 5 (μM) |
|---|---|---|---|
| 0 | 110 | 110 | 110 |
| 2 | 20 | 870 | 870 |
| 4 | 20 | 220 | 870 |
| 6 | 110 | 220 | 435 |
| 8 | 20 | 220 | 870 |
| 10 | N/A | 220 | 220 |
| 12 | N/A | 220 | 435 |

Saliva nitrite at the baseline was very low and stayed flat. With Formula 4, saliva nitrite increased throughout the 12 hours. Saliva nitrite increased by 600% at the peak of 2 hours. At the 12-hour mark, saliva nitrite was still much higher than the initial timepoint and also the baseline. In summary, this advance formula boosted saliva nitrite to a much higher level and sustained the increase in NO bioavailability longer.

In addition, the composition was evaluated for its ability to increase saliva nitrite, to improve endothelial function, and to reduce blood pressure in a mildly hypertensive male over 24 hours (Table VIII, IX, and XI).

At time zero, saliva nitrite levels as measured with Berkeley Test® Nitric Oxide Test Strip are the same for the baseline and Formula 4 (Table VIII). However, saliva nitrite for Formula 4 stayed much higher than the baseline over 24 hours. The increase was 7-fold at 4 and 6 hours. These data demonstrate Formula 4 sustained saliva nitrite and, therefore, a marked increase of NO bioavailability for 24 hours.

TABLE VIII
(Saliva Nitrite)

| Hour | Baseline (μM) | Formula 4 (μM) |
|---|---|---|
| 0 | 110 | 110 |
| 2 | N/A | 435 |
| 4 | N/A | 870 |
| 6 | N/A | 870 |
| 8 | N/A | 435 |
| 10 | N/A | 435 |
| 12 | 110 | 435 |
| 24 | N/A | 110 |

The subject had a mild systolic hypertension (Table IX) and was under no medication. After ingesting Formula 4 in the morning, his systolic blood pressure dropped consistently from 2 hours to 20 hours. In essence, the hypotensive effect of Formula 4 lasted for 24 hours. The formula is indicated for CVD.

TABLE IX
(Blood Pressure)

| | Baseline | Formula 4 |
|---|---|---|
| Hour | (Systolic/Diastolic, mmHg) | (Systolic/Diastolic, mmHg) |
| 0 | 133/73 | 137/67 |
| 2 | N/A | 125/67 |
| 4 | N/A | 133/75 |
| 6 | N/A | 128/65 |
| 8 | N/A | 125/72 |
| 10 | N/A | 119/70 |
| 12 | 135/69 | 120/69 |
| 20 | N/A | 130/73 |
| 24 | N/A | 135/68 |

As discussed earlier, endothelial dysfunction leads to CVD including hypertension. Endothelial function of the subject was evaluated by pulse wave analysis (PWA) with SA-3000P from Medicore. The medical device generates an accelerated plethysmogram (APG) waveform. The waveform pattern can be used to assess the endothelial function and CVD risk. Table X lists the waveform types and corresponding vascular health conditions.

TABLE X
(Waveform Pattern)

| | Type 1 | Type 2 | Type 3&4 | Type 5 | Type 6&7 |
|---|---|---|---|---|---|
| Vessel and | Excellent | Fair but | Poor and | Bad and | Serious |
| Circulation | | start | plaque | significant | plaque |
| | | buildup | buildup | plaque buildup | buildup |

PWA shows the subject had suboptimal circulation and vessel condition with waveform type 4 (Table XI). After ingesting a single serving of Formula 4, the waveform improved in 2 hours. The beneficial effect lasted for at least 12 hours. This is not surprising and confirmed that Formula 4 sustained an increased NO bioavailability for 24 hours.

TABLE XI
(APG Waveform)

| Hour | Baseline (Waveform Type) | Formula 4 (Waveform Type) |
|---|---|---|
| 0 | 4 | 3 |
| 2 | N/A | 2 |
| 4 | N/A | 2 |
| 6 | N/A | 3 |
| 8 | N/A | 2 |
| 10 | N/A | 2 |
| 12 | 4 | 2 |
| 20 | N/A | 4 |
| 24 | N/A | 3 |

Furthermore, we conducted 2 separate experiments to analyzed exhaled air from the lung for NO and H₂S, and also transdermal H₂S. In the first experiments, saliva NO and fractional exhaled NO (FeNO) were measured after a healthy young male adult consumed Formula 4. FeNO was measured in duplicate with Bedfont NOBreath®. As seen in table XII, both saliva NO and breath NO show an increase at 2 hours. This increase lasted for at least another 10 hours for saliva NO and 6 hours for breath NO after dosing. In this case, saliva NO peaked at 4 hours and breath NO peaked earlier at 2 hours. These data demonstrate that Formula 4 also increases breath NO. FeNO seems to respond faster but less dramatic to the formula. It may be used to monitor NO availability in the body for its convenience and sensitivity.

TABLE XII
(Saliva and Breath NO)

| Hour | Saliva NO (μM) | FeNO (ppb) |
|---|---|---|
| 0 | 20 | 17.5 |
| 1 | 20 | 21 |
| 2 | 110 | 31 |
| 3 | 220 | 26 |
| 4 | 435 | 24 |
| 6 | 110 | 22.5 |
| 8 | 110 | 25.5 |
| 10 | 110 | 18.5 |
| 12 | 110 | 14.5 |
| 24 | 20 | 18 |

In the second experiment, exhaled H₂S from the lung was measure in the throat to avoid the interference of H₂S originated in the mouth of a healthy adult male subject. The mouth was also thoroughly washed before each measurement. Separately, the transdermal H₂S on the surface of the forearm was measured on a different day. Exhaled H₂S showed an increased at 2 hours and peaked at 5 hours after dosing (Table XIII). Transdermal H₂S also showed an increase at 5 hours after dosing. Both dropped back to baseline at 6 hours after dosing. The duration of H₂S elevation as indicated by exhaled and transdermal measurements is shorter than the sustained increased of NO by Formula 4. This is also seen with an increase of transdermal H₂S by N-acetyl cysteine in a previous experiment.

This phenomenon may be explained by the interaction of H₂S with NO and some NO precursors/donors to generate S-nitrosothiols (SNOS). SNOS are organic esters of nitrite and sulfhydryl (thiol) groups that act to store, transport, and release NO. They are intermediates in NO-dependent signaling processes that mediate vasodilation and other cardiovascular effects of NO. Stimulating H₂S production and the subsequent SNOS formation contribute to the increased bioavailable of systemic NO and the synergistic effect of NO and H₂S precursors in Formula 4.

TABLE XIII
(Hydrogen Sulfide or Volatile Sulfur Compounds)

| Hour | Exhaled (ppb) | Transdermal (ppb) |
|---|---|---|
| 0 | 185 | 25 |
| 1 | 148 | 27 |
| 2 | 219 | 19 |
| 3 | 165 | 21 |
| 4 | 259 | 25 |
| 5 | 340 | 37 |
| 6 | 208 | 16 |

To fully evaluate the efficacy of Formula 4 to stimulate NO and its vasodilating effect, we conducted an open label human study in 12 subjects with mild hypertension at a research medical clinic. Each subject took a single serving of Formula 4 once in the morning every day for 4 weeks. Saliva nitrite was measured pre-dosing and 2 hours after dosing with Berkeley Test® Nitric Oxide Test Strip and blood pressures were measured in triplicate during Visit 1 (baseline), Visit 2 (2 weeks) and Visit 3 (4 weeks) at the clinic. Each subject also measured blood pressures once in the morning and saliva nitrite 3 times (pre-dosing in the morning, 2 hours and 6 hours after-dosing) every day at home for 4 weeks.

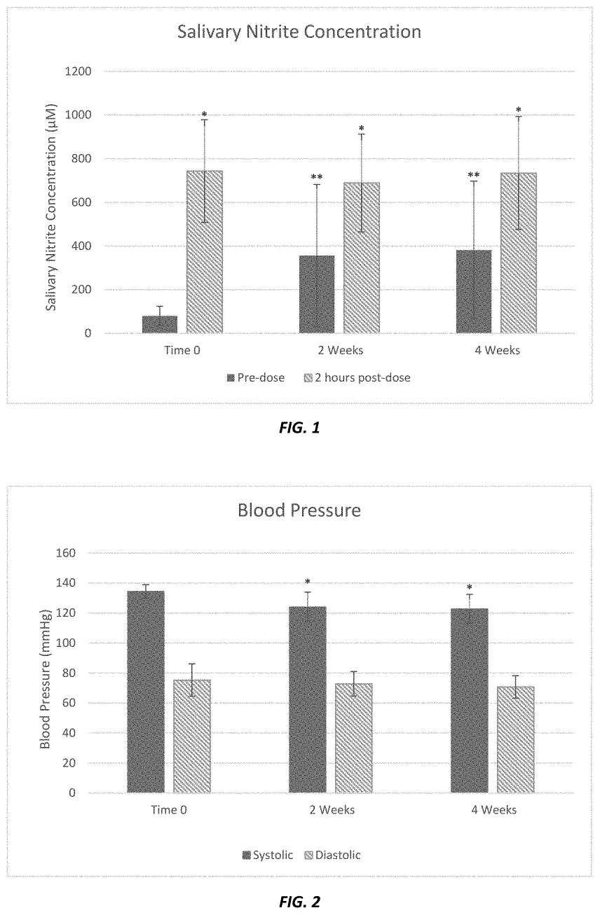

During Visit 1, saliva nitrite of subjects was measured with Berkeley Test® Nitric Oxide Test Strip and data are presented in Table XIV and FIG. 1. The average saliva nitrite was 80.0 μM before ingesting Formula 4. The number significantly increased to 743.3 μM 2 hours after ingesting Formula 4 (p=0.0000004). After 2 weeks, the average saliva nitrite before ingesting Formula 4 was 356.3 μM during Visit 2. This number is markedly higher than 80.0 μM 2 weeks ago. The increase reaches a statistical significance at a p-value of 0.01. The elevated saliva nitrite before ingesting Formula 4 after 2 weeks indicates the daily dose of Formula 4 has a lasting effect on increasing NO bioavailability for more than 24 hours. Two hours after ingesting Formula 4 during visit 2, average saliva nitrite further climbed to 688.8 μM, a significant 8-fold increase compared to the original level at 80.0 μM. After 4 weeks, the average saliva nitrite before ingesting Formula 4 was 381.7 μM during Visit 3. This number is similar to 356.3 μM from 2 weeks ago but significantly higher than 80.0 μM 4 weeks ago (p=0.005). Two hours later, saliva nitrite reached 734.2 μM, a similar increase in 2 hours also seen during Visit 1 and 2. Patient self-reported data are in good agreement with the data collected in the clinic. It is worth noting that average saliva nitrite was 580.4 μM 6 hours after dosing on the day of Visit 1 as reported by the patients. The day after Visit 1, saliva nitrite levels were 348.3 μM, 616.7 μM and 321.7 μM at pre-dosing, 2 hours and 6 hours after dosing, respectively. In addition, the ranges of average saliva nitrite reported by patients were 312.1-535.0 μM, 490.0-761.3 μM, and 453.8-707.1 μM at pre-dosing, 2 hours and 6 hours after dosing, respectively during the 4 weeks. Research data indicate saliva nitrite at above 220 μM is considered the minimum therapeutic concentration associated with a hypotensive benefit. This is normally achieved with a higher level of NO precursor nitrate than is present in Formula 4. However, such a saliva nitrite level usually lasts anywhere from less than an hour to a few hours with current products on the market. On the other hand, Formula 4 surpassed 220 μM significantly within 2 hours and sustains the level by a large margin for at least 24 hours after a single dosing. It is also important to note that repeated daily dosing with Formula 4 for 4 weeks did not cause a continuing increase of saliva nitrite. Saliva nitrite levels stayed relatively stable. Again, these data clearly demonstrate the Formula 4 increases bioavailable NO with both a fast and a long-lasting action.

TABLE XIV
(Saliva NO₂)

| | 0 hrs (μM, | 2 hrs (μM, | |
|---|---|---|---|
| Saliva Nitrite | Mean ± STDEV) | Mean ± STDEV) | p-Value |
| Visit 1 | 80.0 ± 44.3 | 743.3 ± 235.2 | 0.0000004 |
| (Baseline) | | | |
| Visit 2 (2 weeks) | 356.3 ± 325.8 | 688.8 ± 224.0 | 0.0084061 |
| Visit 3 (4 weeks) | 381.7 ± 315.5 | 734.2 ± 258.4 | 0.0027351 |

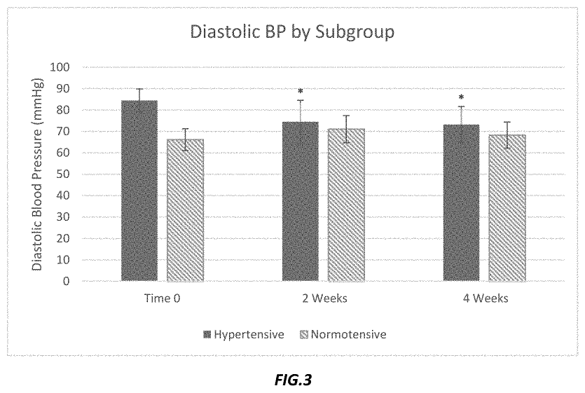

Patients were under medical care and their hypertension being treated. However, their systolic blood pressure was not under control at an average of 134.4 mmHg before a single daily dosing with Formula 4 (Table XV and FIG. 2). After 2 weeks, the average systolic blood pressure dropped more than 10 points to 124.1 mmHg. Basically, treatment of Formula 4 for 2 weeks brought their systolic blood pressure under control and into a normal range. The significant reduction has a p-value of 0.0014. After 4 weeks, the average systolic blood pressure further dropped by 1.3 points to 122.8 mmHg. A total drop of 11.6 points of systolic blood pressure over 4 weeks is statistically significant with a p-value of 0.0003. At the same time, the average diastolic pressure dropped by more than 2.5 points from 75.3 to 72.8 mmHg in 2 weeks and a total of 4.5 points to 70.8 mmHg in 4 weeks. Although a 4.5-point reduction of diastolic pressure in 4 weeks is impressive by any standard, it did not reach the statistical significance with a p-value of 0.083. We further analyzed the diastolic blood pressure data by dividing the patients into two subgroups. One subgroup included the 6 patients with an initial diastolic blood pressure equal to or higher than 80 mmHg (hypertensive diastolic pressure). Another subgroup included the 6 patients with diastolic blood pressure less than 80 mmHg (normotensive diastolic pressure). The data are presented in Table XVI and FIG. 3. For the patients with diastolic hypertension, their average diastolic blood pressure dropped significantly by 9.9 points from 84.4 to 74.5 mmHg in the first 2 weeks (p-value=0.008). There was another drop of 1.3 points by the end of 4 weeks and the total reduction of diastolic pressure was 11.2 mmHg with a p-value of 0.001. On the other hand, there was no significant change in diastolic blood pressure for patients with normal diastolic blood pressure during the 4 weeks. These hypertension data clearly demonstrate Formula 4 affected a long-lasting effect on NO bioavailability which led to a hypotensive benefit. It significantly reduced blood pressures in hypertensive patients within 2 weeks. However, it does not seem to lower blood pressures when they are in the normal ranges.

TABLE XV
(Blood Pressure)

| Blood | Systolic Pressure (mmHg, | Diastolic Pressure (mmHg, |
|---|---|---|
| Pressure | Mean ± STDEV) | Mean ± STDEV) |
| Visit 1 | 134.4 ± 4.49 | 75.3 ± 10.77 |
| Visit 2 | 124.1 ± 9.89 | 72.8 ± 8.18 |
| Visit 3 | 122.8 ± 9.69 | 70.8 ± 7.47 |

TABLE XVI
(Diastolic Blood Pressure)

| | Hypertensive | Normotensive |
|---|---|---|
| | (mmHg, Mean ± STDEV) | (mmHg, Mean ± STDEV ) |
| Visit 1 (Baseline) | 84.4 ± 5.37 | 66.2 ± 5.11 |
| Visit 2 (2 Weeks) | 74.5 ± 9.99 | 71.1 ± 6.35 |
| Visit 3 (4 weeks) | 73.2 ± 8.45 | 68.3 ± 6.06 |

### Example 5—An Advanced NO Precursor with H₂S Precursor Composition with an eGRC (Formula 5)

The composition was prepared using the components set forth in Table XVII (Formula 5), by adding the eGRC MNE in the composition of Example 4 (Formula 4). MNE repairs, regenerates and restores the endothelial glycocalyx (EGX). A healthy EGX enables eNOS to synthesize NO. It also houses extracellular superoxide dismutase (ecSOD) that controls oxidative stress by ROS at the endothelium and makes more NO available for its biological function. Formula 5 has the advantage of additional synergistic effects to boost NO production and availability.

TABLE XVII
(Formula 5)

| Function | Ingredient | Amount (mg/serving) | |
|---|---|---|---|
| NO Precursor | Potassium Nitrate | 380 | mg |
| NO Precursor | Beetroot Extract | 200 | mg |
| H₂S Precursor | Fermented Black | 400 | mg |
| | Garlic Extract (SAC) | | |
| eGRC | MNE | 300 | mg |
| Antioxidant and reducing | Ascorbic Acid | 180 | mg |
| agent to facilitate NO | (Vitamin C) | | |
| metabolism | | | |
| Plant polyphenols and | Bilberry Extract | 100 | mg |
| regulating NO | | | |
| bioavailability | | | |
| Organosulfur NO donor | Thiamine Mononitrate | 80 | mg |
| | (Vitamin B1) | | |
| Increase NO and NO- | Magnesium | 56 | mg |
| dependent vasodilation | | | |
| Enhance eNOS function | Zinc | 3 | mg |
| Support eNOS cofactor | Methyl Cobalamin | 100 | mcg |
| BH4 | (Vitamin B12) | | |
| Increase/regulate eNOS | Cholecalciferol | 60 | mcg |

| | |
|---|---|
| and SOD expression | (Vitamin D3) |

The composition was evaluated for its ability to increase NO production and availability. A healthy young male ingested a single serving of Formula 5 in the morning only and his saliva nitrite (NO₂) was determined with MyFitStrip® Saliva NO Test Strip for 12 hours. As seen in Table VII, saliva nitrite with Formula 5 was significantly higher than the baseline throughout the period. In fact, 4 of the 7 data points during the 12 hours are also higher than those with Formula 4 at 4, 6, 8 and 12 hours. These data clearly show an overall higher NO bioavailability sustained by Formula 5 compared with that of Formula 4. It is also observed that such an effect goes far beyond 12 hours. Based on the 24-hour effect of Formula 4 demonstrated by other data, Formula 5 is expected to sustain a higher NO bioavailability for at least 24 hours. The data indicate Formula 5 is even better than Formula 4 as an innovative therapeutic composition to increase vascular NO for the treatment of ED and related condition such as hypertension.

It is understood that the above-described various types of compositions, dosage forms and/or modes of applications are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that variations including, but not limited to, variations in size, quantities, materials, shape, form, function and manner of operation, assembly and use may be made without departing from the principles and concepts set forth herein.

What is claimed is:

1. A therapeutic composition for treating endothelial dysfunction, comprising:
a combination of a nitric oxide (NO) precursor and a hydrogen sulfide (H₂S) precursor in an amount sufficient to sustain an increase of bioavailable NO for treatment of endothelial dysfunction; and
a pharmaceutically acceptable carrier.

2. The therapeutic composition of claim 1, wherein the NO precursor comprises L-arginine, L-arginine alpha-ketoglutarate, L-citrulline, or combinations thereof.

3. The therapeutic composition of claim 1, wherein the NO precursor comprises inorganic nitrite, inorganic nitrate, organic nitrate, or combinations thereof.

4. The therapeutic composition of claim 1, wherein the NO precursor comprises nitrite or nitrate salts of sodium, potassium, calcium, magnesium, manganese, iron, copper, chromium, zinc, or combinations thereof.

5. The therapeutic composition of claim 1, wherein the NO precursor is derived from arugula, celery, cress, lettuce, chervil, beetroot, spinach, mustard greens, cabbage, fennel, leek, parsley, rocket, swiss chard, leafy chicory, kohlrabi, radish, or combinations thereof.

6. The therapeutic composition of claim 1, wherein the NO precursor is derived from danshen root (Radix salvia miltorrhizae), snakegourd fruit (Fructus trichosanthis), longstamen onion bulb (Bulbus allii macrostemi), sanchi (Radix notoginseng), ginseng (Radix ginseng), borneol (Borneolum syntheticum), and borneol (Cinnamomum), or combinations thereof.

7. The therapeutic composition of claim 1, wherein the NO precursor is a blend of powdered extracts.

8. The therapeutic composition of claim 1, wherein the NO precursor is a blend of liquid extracts.

9. The therapeutic composition of claim 1, wherein the NO precursor is present in the composition in an amount of from about 10 wt % to about 90 wt %.

10. The therapeutic composition of claim 1, wherein the hydrogen sulfide precursor comprises sodium hydrosulfide (NaHS), sodium sulfide, N-acetyl cysteine (NAC), S-allyl cysteine (SAC), glutathione (GSH), a garlic-derived organic polysulfide, a sulfated oligosaccharide, a sulfated polysaccharide, a natural isothiocyanate, other organosulfur compounds (OSCs), synthetic H₂S donors such as phosphorodithioate derivatives, NOSH compounds which act as both NO and H₂S precursors, or combinations thereof.

11. The therapeutic composition of claim 1, wherein the hydrogen sulfide precursor is present in the composition in an amount of from about 10 wt % to about 90 wt %.

12. The therapeutic composition of claim 1, further comprising an antioxidant.

13. The therapeutic composition of claim 12, wherein the antioxidant comprises antioxidant enzymes of superoxide dismutase (SOD), catalase, glutathione peroxidase, or combinations thereof, or non-enzymatic antioxidants of NAC, glutathione, vitamin C, lipoic acid, polyphenols, carotenoids, vitamin E, or combinations thereof.

14. The therapeutic composition of claim 12, wherein the antioxidant is present in the composition in an amount of from about 1 wt % to about 80 wt %.

15. The therapeutic composition of claim 1, further comprising an endothelial glycocalyx regenerator.

16. The therapeutic composition of claim 15, wherein the glycocalyx regenerator comprises a sulfated polysaccharide, a sulfated oligosaccharide, chito-oligosaccharides (COS), or combinations thereof.

17. The therapeutic composition of claim 15, wherein the glycocalyx regenerator comprises rhamnan sulfate, fucoidan sulfate, carrageenan, or combinations thereof.

18. The therapeutic composition of claim 15, wherein the glycocalyx regenerator is present in the composition in an amount of from about 10 wt % to about 80 wt %.

19. The therapeutic composition of claim 1, wherein the pharmaceutically acceptable carrier comprises water, a solubilizing agent, a dispersing agent, a tonicity agent, a pH adjuster, a buffering agent, a preservative, a chelating agent, a bulking agent, a binder, a disintegrant, a filler, a glidant, a lubricant, a sweetener, a thickening agent, eNOS cofactors, or a combination thereof, wherein cofactors are at least one of magnesium, zinc, pyridoxine (B6), folic acid (B9), vitamin B12, vitamin C, vitamin D, and tetrahydrobiopterin (BH4).

20. The therapeutic composition of claim 1, wherein the NO precursor includes two NO precursors as potassium nitrate (at 100-500 mg/serving) and beetroot extract (at 50-2000 mg/serving), the H₂S precursor is fermented black garlic extract (at 100-1000 mg/serving) and vitamin B1 (at 10-200 mg/serving), and the composition further comprises an endothelial glycocalyx regenerating compound which is Monostroma nitidum extract (at 25-500 mg/serving), a nonenzymatic antioxidant which is vitamin C (at 30-300 mg/serving), a plant-based antioxidant which is bilberry extract (at 50-500 mg/serving), and multiple eNOS cofactors which include magnesium (at 10-200 mg/serving), zinc (at 1-30 mg/serving), methyl cobalamin (at 10-200 mcg/serving), and cholecalciferol (at 2.5-100 mcg/serving).

21. A method of treating endothelial dysfunction in a subject, comprising:
administering to the subject a combination of a nitric oxide (NO) precursor and a hydrogen sulfide (H₂S) precursor in an amount and at a frequency sufficient to stabilize and reverse damage in the endothelial glycocalyx (EGX).

22. The method of claim 21, wherein administering is performed orally or via injection.

23. The method of claim 21, wherein the NO precursor comprises L-arginine, L-arginine alpha-ketoglutarate, L-citrulline, or combinations thereof.

24. The method of claim 21, wherein the NO precursor comprises inorganic nitrite, inorganic nitrate, organic nitrate, or combinations thereof.

25. The method of claim 21, wherein the NO precursor comprises nitrite or nitrate salts of sodium, potassium calcium, magnesium, manganese, iron, copper, chromium, zinc, or combinations thereof.

26. The method of claim 21, wherein the NO precursor is derived from arugula, celery, cress, lettuce, chervil, beetroot, spinach, mustard greens, cabbage, fennel, leek, parsley, rocket, swiss chard, leafy chicory, Kohlrabi, radish, or combinations thereof.

27. The method of claim 21, wherein the NO precursor is derived from danshen root (Radix salvia miltorrhizae), snakegourd fruit (Fructus trichosanthis), longstamen onion bulb (Bulbus allii macrostemi), sanchi (Radix notoginseng), ginseng (Radix ginseng), borneol (Borneolum syntheticum), and borneol (Cinnamomum), or combinations thereof.

28. The method of claim 21, wherein the NO precursor is a blend of powdered extracts.

29. The method of claim 21, wherein the NO precursor is a blend of liquid extracts.

30. The method of claim 21, wherein the NO precursor is administered in an amount from about 50 mg to about 1000 mg per dose.

31. The method of claim 21, wherein administering is performed at a frequency of once or twice daily.

32. The method of claim 21, wherein the hydrogen sulfide precursor comprises sodium hydrosulfide (NaHS), sodium sulfide, N-acetyl cysteine (NAC), S-allyl cysteine (SAC), glutathione (GSH), a garlic-derived organic polysulfide, a sulfated oligosaccharide, a sulfated polysaccharide, a natural isothiocyanate, other organosulfur compounds (OSCs), synthetic H₂S donors such as phosphorodithioate derivatives, NOSH compounds, or combinations thereof.

33. The method of claim 21, wherein the hydrogen sulfide precursor is administered in an amount from about 10 mg to about 2000 mg per dose.

34. The method of claim 21, further comprising identifying EGX or endothelial dysfunction of the subject.

35. The method of claim 34, wherein identifying comprises at least one of biomarkers, Alpha Elution Technology, dark field microscopy, coronary epicardial vasoactivity, flow-mediated dilation, plethysmography, and EndoPat.

36. The method of claim 21, further comprising administering an antioxidant.

37. The method of claim 21, further comprising administering a glycocalyx regenerator.

38. An oral dosage form, comprising:
a combination of a nitric oxide (NO) precursor and a hydrogen sulfide (H₂S) precursor in an amount sufficient to treat endothelial dysfunction; and
a pharmaceutically acceptable carrier.

39. The oral dosage form of claim 38, wherein the NO precursor comprises L-arginine, L-arginine alpha-ketoglutarate, L-citrulline, or combinations thereof.

40. The oral dosage form of claim 38, wherein the NO precursor comprises inorganic nitrite, inorganic nitrate, organic nitrate, or combinations thereof.

41. The oral dosage form of claim 38, wherein the NO precursor comprises nitrite or nitrate salts of sodium, potassium, calcium, magnesium, manganese, iron, copper, chromium, zinc, or combinations thereof.

42. The oral dosage form of claim 38, wherein the NO precursor is derived from arugula, celery, cress, lettuce, chervil, beetroot, spinach, mustard greens, cabbage, fennel, leek, parsley, rocket, swiss chard, leafy chicory, Kohlrabi, radish, or combinations thereof.

43. The oral dosage form of claim 38, wherein the NO precursor is derived from danshen root (Radix salvia miltorrhizae), snakegourd fruit (Fructus trichosanthis), longstamen onion bulb (Bulbus allii macrostemi), sanchi (Radix notoginseng), ginseng (Radix ginseng), borneol (Borneolum syntheticum), and borneol (Cinnamomum), or combinations thereof.

44. The oral dosage form of claim 38, wherein the NO precursor is administered in an amount from about 50 mg to about 1000 mg per dose.

45. The oral dosage form of claim 38, wherein the hydrogen sulfide precursor comprises sodium hydrosulfide (NaHS), sodium sulfide, N-acetyl cysteine (NAC), S-allyl cystine (SAC), glutathione (GSH), a garlic-derived organic polysulfide, a sulfated oligosaccharide, a sulfated polysaccharide, a natural isothiocyanate, other organosulfur compounds (OSCs), synthetic H₂S donors such as phosphorodithioate derivatives, NOSH compounds, or combinations thereof.

46. The oral dosage form of claim 38, wherein the oral dosage form is formulated as a solid oral dosage form.

47. The oral dosage form of claim 46, wherein the pharmaceutically acceptable carrier of the solid oral dosage form comprises a filler, a binder, a glidant, a disintegrating agent, a lubricant, or a combination thereof.

48. The oral dosage form of claim 46, further comprising an exterior coating.

49. The oral dosage form of claim 38, wherein the oral dosage form is formulated as a liquid oral dosage form.

50. The oral dosage form of claim 49, wherein the pharmaceutically acceptable carrier of the liquid oral dosage form comprises a solubilizing agent, a dispersing agent, a thickener, a sweetener, a pH adjuster, a buffering agent, a tonicity agent, a preservative, or a combination thereof.

51. The oral dosage form of claim 38, further comprising an antioxidant.

52. The oral dosage form of claim 38, further comprising a glycocalyx regenerator.
